(19) United States
(12) Patent Application Publication
(10) Pub. No.: US 2017/0105966 A1
(43) Pub. Date: Apr. 20, 2017

(54) PHARMACEUTICAL COMPOSITION CONTAINING INDOMETACIN AND/OR ACEMETACIN

(71) Applicants: Roger Imboden, Munchenstein (CH); Eric Rothenbuhler, Oberwil (CH); Juerg Lutz, Binningen (CH)

(72) Inventors: Roger Imboden, Munchenstein (CH); Eric Rothenbuhler, Oberwil (CH); Juerg Lutz, Binningen (CH)

(21) Appl. No.: 15/392,418

(22) Filed: Dec. 28, 2016

Related U.S. Application Data
(63) Continuation of application No. 11/997,348, filed on Feb. 28, 2008, now abandoned.

(51) Int. Cl. A61K 31/405; A61K 9/14; A61K 47/26; A61K 9/02; A61K 9/00; A61K 9/46; A61K 9/20; A61K 9/48

(52) U.S. Cl. CPC A61K 31/405; A61K 9/20; A61K 9/141; A61K 9/48; A61K 9/02; A61K 9/0019; A61K 9/0007; A61K 9/4825; A61K 9/0053; A61K 9/2018; A61K 9/145; A61K 9/4858; A61K 47/26

(57) ABSTRACT

The invention relates to a pharmaceutically active composition or pharmaceutical form of administering that contains at least one of the active ingredients indomethacin or acemetacin and optionally other adjuvants, the composition containing the active ingredient, or a mixture of the active ingredients, in micronized form, preferably mixed with at least one flavonoid derivative or a polypeptide or with a mixture of such compounds

This is a continuation application of U.S. patent application Ser. No. 11/997,348, filed on Feb. 28, 2008. The disclosure of that application is expressly incorporated herein by reference.

The present patent application relates to pharmaceutical compositions or forms of administration containing at least one of the active ingredients indomethacin and acemetacin in micronized form. The present invention further relates to processes for the preparation of these compositions.

The compounds indomethacin and acemetacin and their preparation are known. The compounds have anti-inflammatory, pain-relieving and antipyretic properties. Indomethacin has the chemical nomenclature 1-(p-chlorobenzoyl)-5-methoxy-2-methyl-3-indolylacetic acid. Acemetacin has the chemical nomenclature 1-(p-chlorobenzoyl)-5-methoxy-2-methyl-3-indolylacetoxyacetic acid.

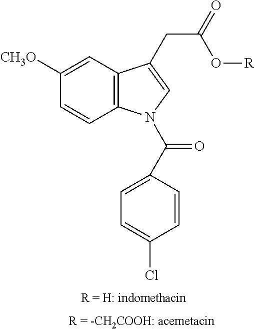

Said active ingredients, especially acemetacin, are hydrophobic, poorly wettable and also poorly soluble in solvents suitable for pharmaceutical use. Moreover, both active ingredients, especially acemetacin, have a bitter taste, making them unsuitable for peroral administration, e.g. in the form of an effervescent preparation. Unexpectedly, the bitter taste, e.g. in an effervescent preparation or a suspension, could not be masked with the flavourings conventionally used in pharmacy. However, when said active ingredients are used, e.g. for conditions of chronic and, in particular, acute pain, it is of great advantage to have the fastest possible release of the active ingredient, or the rapid attainment of a high blood level, and sometimes also to administer it perorally, e.g. in the form of an effervescent preparation.

U.S. Pat. No. 4,687,762 describes the preparation of water-soluble complexes of water-insoluble, pharmaceutically active compounds, e.g. acemetacin, wherein the pharmaceutically active compound is dissolved in an organic solvent and brought into contact with a phospholipid. This process has the disadvantage that an organic solvent has to be used, so the original crystalline form is dissolved to give a liposomal formulation in the form of semisolid vesicles. U.S. Pat. No. 5,932,245 describes a colloidal dispersion prepared with a selected gelatin, inter alia for acemetacin and indomethacin, in the form of a nanosol.

It has now been found that the micronized form of the active ingredients indomethacin and acemetacin, especially acemetacin, only exhibits said disadvantages to a reduced extent, if at all. Surprisingly, these active ingredients can thus be administered perorally in quick-dissolving or quick-releasing, galenical forms, especially effervescent preparations or suspensions, or in quick-dissolving, solid dosage forms such as tablets or capsules, which rapidly gives blood levels that are sufficiently high for medicinal use, this being advantageous especially in the treatment of conditions of chronic and, in particular, acute pain. This is coupled with the further advantage that, in the case of effervescent preparations, the release of carbon dioxide in the stomach stimulates the stomach walls and initiates peristaltic movements, leading to a desirable rapid evacuation of the solution or suspension of the effervescent preparation into the duodenum.

It has also been found, surprisingly, that the bitter taste of micronized indomethacin and acemetacin is efficiently masked by the addition of flavonoid derivatives, e.g. neohesperidin dihydrochalcone, and/or polypeptides, e.g. thaumatin, even in small amounts. It is also surprising that, despite the large surface area of the micronized active ingredient and the increased gustatory activity associated therewith, an efficient masking of the taste is achieved with comparatively small concentrations of flavonoid derivatives and polypeptides.

The present invention relates to a pharmaceutically effective composition or pharmaceutical form of administration containing at least one of the active ingredients indomethacin and acemetacin and optionally other additives, characterized in that this composition contains the active ingredient or a mixture of these active ingredients, i.e. indomethacin and/or acemetacin, in micronized form. The present invention further relates to an above-defined composition or pharmaceutical form of administration which is characterized in that it contains the active ingredient(s) in a mixture with at least one flavonoid derivative or a polypeptide or a mixture of such compounds.

The present invention further relates to a process for the preparation of the composition according to the invention, characterized in that, prior to production of the form of administration, the active ingredient(s) is (are) micronized, preferably using mechanical means, i.e. mechanically micronized, after which the form of administration is produced using the micronized active ingredient(s).

The present invention further relates to the use of the composition according to the invention for the treatment of pain conditions, inflammations and fever, especially chronic polyarthritis, degenerative joint diseases, especially of the large joints and the spinal column, Bechterew's disease, gout, inflammatory conditions of the joints, muscles and tendons, tendovaginitis, bursitis, lumbago and superficial venous inflammations (thrombophlebitis).

The present invention further relates to the active ingredients indomethacin and acemetacin as bulk powders, optionally in a mixture with other additives, characterized in that the active ingredients are in micronized form.

The present invention further relates to the active ingredients indomethacin and acemetacin as bulk powders, optionally in a mixture with other additives, characterized in that the active ingredients are in micronized form in a mixture with at least one flavonoid derivative or a polypeptide or a mixture of such compounds.

The present invention further relates to the use of the active ingredients indomethacin and acemetacin in micronized form as bulk powders, optionally in a mixture with a flavonoid derivative or a polypeptide or a mixture of such compounds, and optionally in a mixture with other additives, for the production of the normal-releasing and quick-releasing forms of administration according to the invention, or of pharmaceutical compositions, especially for the medicinal treatment of pain conditions, inflammations and fever, especially chronic polyarthritis, degenerative joint diseases, especially of the large joints and the spinal column, Bechterew's disease, gout, inflammatory conditions of the joints, muscles and tendons, tendovaginitis, bursitis, lumbago and superficial venous inflammations (thrombophlebitis).

The claimed pharmaceutical compositions or pharmaceutical forms of administration comprise especially (i) tablets such as peroral tablets, chewing tablets, oral tablets (sucking tablets, sublingual tablets, buccal tablets), parenteral tablets, dissolving tablets and effervescent tablets; (ii) capsules such as hard gelatin capsules and soft gelatin capsules; (iii) liquid dosage forms such as solutions, emulsions and suspensions; (iv) rectal products, preferably suppositories such as suspension suppositories and dissolving suppositories, or rectal capsules; and (v) preparations for parenteral administration, preferably intramuscular or subcutaneous administration.

For the purposes of the present invention, “micronized” denotes a very fine particle size in the micrometre range, these micronized particles preferably being produced according to the invention by mechanical means and their crystal structure remaining unchanged.

Preferably, the expression “active ingredient in micronized form” denotes that at least 90% by volume, preferably at least 95% by volume and particularly preferably at least 98% by volume of the active ingredient has a particle size below 25 μm (micron) (<25 μm) and preferably below 21 μm (micron) (<21 μm). For these purposes, the “active ingredient in micronized form” is preferably in the form of microcrystals with which highly disperse systems can also be produced.

Preferably at least 50% by volume and particularly preferably at least 60% by volume of the active ingredient has a particle size below 10 μm (micron) (<10 μm) and preferably below 8 μm (micron) (<8 μm).

Preferably at least 30% by volume of the active ingredient and particularly preferably at least 50% by volume of the active ingredient has a particle size below 5 μm (micron) (<5 μm). The lower particle size limit is about 1 μm (micron) for all said values. The micronized active ingredient is used directly either as micronized powder or in processed form, e.g. as pellets or granules, or is processed further.

Methods of micronizing active ingredients are known per se. Examples of such methods are dry grinding in a ball mill or jet mill, and wet grinding in an agitated ball mill (bead mill, sand mill) or colloid mill, such as those described in the relevant scientific literature. The apparatuses which can be used for micronization are commercially available, e.g. the Chrispro® Jet-Mill MC 300KX-TD apparatus from Micro-Macinazione SA. Jet milling is preferred.

Examples of flavonoid derivatives are those belonging to the group comprising chalcones and dihydrochalcones or their glycosides, and combinations and complexes prepared therefrom, especially chalcones and dihydrochalcones and glycosides derived therefrom, i.e. chalcone glycosides and dihydrochalcone glycosides. Such compounds are known per se. Typical representatives are e.g. naringin chalcone (R=glycoside, 3,4-unsaturated) or hesperetin dihydrochalcone glucoside (R=glucoside) and, in particular, neohesperidin dihydrochalcone (R=glycoside).

Dihydrochalcones have the formula below:

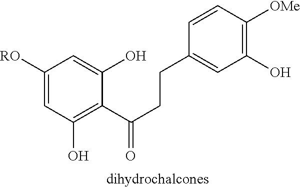

in which R is a radical known per se, preferably hydrogen or (C₁₋₆)-alkyl. R is preferably a radical corresponding to the dihydrochalcone compounds listed below.

The weight ratio of active ingredient to flavonoid compound ranges preferably from 10:1 to 50:1, particularly preferably from 2:1 to 50:1 and especially from 1:1 to 15:1. In the case of neohesperidin dihydrochalcone, it is especially about 6:1. The active ingredient(s) is (are) mixed with the flavonoid compound or a mixture of such compounds, the active ingredient optionally being microencapsulated in a manner known per se.

Examples of oligopeptides and polypeptides and derivatives thereof which are optionally present in the mixture are particularly dipeptides, such as dipeptides derived from L-aspartic acid and dipeptide esters derived from L-aspartic acid, and dipeptides and dipeptide esters derived from L-aminomalonic acid, especially L-aspartyl-D-alanine, L-aspartyl-L-phenylalanine methyl ester and L-aspartyl-L-methionine methyl ester, and dipeptides and dipeptide esters derived from lysine, especially N-phenylacetylglycyllysine and N-acetylphenylalanyllysine, and polypeptides and polypeptide mixtures obtained from tropical plants and having a molecular weight of between 5 and 100 kDa and a high sweetening power, such as brazzein (monomer: about 54 amino acids), curculin (monomer: about 114 amino acids), mabinlin (dimer: about 33+about 72 amino acids), miraculin (tetramer: about 191 amino acids), monellin (dimer: about 45+about 50 amino acids), pentadin and thaumatin (about 207 amino acids).

The weight ratio of active ingredient to oligopeptides and/or polypeptides ranges from 10:1 to 100:1, preferably from 2:1 to 100:1 and especially from 1:1 to 20:1. In the case of thaumatin, it is especially about 6:1. The active ingredient(s) is (are) mixed with the peptide or a mixture of such compounds, the active ingredient optionally being microencapsulated in a manner known per se.

If the composition contains a mixture of flavonoid and polypeptide, the mixing ratio flavonoid:polypeptide is preferably 3:1 to 1:3, particularly preferably 2:1 to 1:2 and very particularly preferably in the range of 1:1. Thus flavonoid:polypeptide mixing ratios of 1:3, 1:2, 1:1, 2:1 or 3:1 present no problems, the total amount of flavonoid+polypeptide corresponding to the amount indicated above for only one of the two components.

Tablets, such as peroral tablets, chewing tablets, oral tablets (sucking tablets, sublingual tablets, buccal tablets), parenteral tablets, dissolving tablets and effervescent tablets, are produced by techniques known per se, such as direct tableting or tableting after the prior production of granules or pellets. Conventional additives that are inert in the composition can be used in this process.

The following are examples of additives which can be used for these granules, pellets and tablets: fillers, such as types of starch known per se, lactose, celluloses, mannitol and sorbitol; binders, such as starches known per se, celluloses and polyethylene glycols; disintegrants, such as starches known per se which can be used per se for this purpose, celluloses, alginates, polyvinylpyrrolidones and sodium hydrogen carbonate; lubricants, such as stearates like magnesium stearate; flow regulators, such as silicon dioxide; and film-forming agents known per se.

Starches which can be used are any of those known per se, rice, wheat and potato starches being preferred. Likewise, modified starches known per se can be used, such as those modified by methyl, hydroxymethyl and/or hydroxypropyl. They are preferably used in proportions of about 5-20%, based on the total weight of the form of administration.

Celluloses which can be used are unmodified pulverulent cellulose, micro-crystalline cellulose or modified cellulose, such as celluloses modified by methyl, ethyl, propyl, hydroxymethyl, hydroxyethyl and/or hydroxypropyl, e.g. HPMC. They can preferably be used in the range from 20 to 90% by weight, based on the total weight of the form of administration, or else used as coating materials.

Silicon dioxide is preferably used in colloidal form in amounts of about 0.1 to 0.5%, based on the weight of all the additives. Lactose can be used in untreated form or, for example, spray-dried in amounts ranging from about 65 to 85%, based on the total weight of the additives. Polyethylene glycols and derivatives thereof are used as lubricants or binders in amounts of 0.5-5% by weight, based on the total weight of the additives. Polyvinylpyrrolidones in amounts of 0.5-5% by weight, based on the total weight of the additives, can be used as binders, disintegrants or coating agents. Calcium hydrogen phosphates and mannitol are used as diluents in the range from 10 to 90% by weight, based on the total weight of the additives. Stearates, preferably magnesium stearate, are used as lubricants in the range from 0.25 to 5% by weight, based on the total weight of all the additives. The production of effervescent preparations, e.g. effervescent tablets, is known per se. They can contain the additives previously mentioned for the production of tablets, but additionally contain an effervescent substance. This effervescent substance usually consists of a combination of a carbonate or hydrogen carbonate, preferably sodium hydrogen carbonate, on the one hand, and a suitable organic acid, preferably citric acid or ascorbic acid, on the other. Such additives are known per se as effervescent substances and are described in numerous compositions.

The composition of the effervescent substance is not critical for the present invention. An effervescent substance consisting of sodium hydrogen carbonate and citric acid and/or ascorbic acid is preferred.

For this purpose, the present invention further relates to an effervescent preparation, preferably an effervescent tablet, which contains at least one of the active ingredients indomethacin and acemetacin, preferably acemetacin, and other additives known per se for an effervescent preparation, characterized in that this composition contains the active ingredient(s), i.e. indomethacin and/or acemetacin, in micronized form and preferably in a mixture with at least one flavonoid derivative or a polypeptide or a mixture of such compounds.

Said effervescent preparation is also covered by the definitions indicated above in respect of the particle size distribution of the micronized active ingredient(s) and their preparation, and in respect of the flavonoid derivative or the polypeptide, and the other additives, it being possible for these active ingredients in the effervescent preparation, preferably in the effervescent tablet, to be in pulverulent form or in modified-release form, e.g. as pellets or as pellets provided with a film coating.

A preferred effervescent preparation, preferably an effervescent tablet, is one which consists of (a) active ingredient granules containing the micronized active ingredient, the latter being (b) in a mixture with at least one flavonoid derivative or a polypeptide or a mixture of such compounds, (c) an effervescent substance consisting of at least one siliconized inorganic carbonate compound or bicarbonate compound, such as siliconized sodium hydrogen carbonate and/or siliconized calcium carbonate, and an organic acid, preferably citric acid, and optionally (d) other additives.

Additives are selected e.g. from the group comprising sweeteners, synthetic sugar substitutes and salts thereof, polyols, natural and synthetically prepared flavourings, loading agents, surfactants, colourants, fillers and binders.

Examples of additives are sweeteners, such as polysaccharides, e.g. sucrose, fructose, glucose, dextrose, isomaltose, inulin, lactitol and trehalose; synthetic sugar substitutes and salts thereof, such as xylitol, cyclamate, acesulfame, sucralose, saccharin, alitame and aspartame; polyols, e.g. glycerol, mannitol and vanillin; and lemon flavouring, strawberry flavouring and natural and synthetically prepared flavourings known per se. The active ingredient granules can contain a loading agent, e.g. sodium sulfate and/or barium sulfate; a surfactant, e.g. a polyoxyethylene castor oil derivative such as macrogol glycerol hydroxystearate, a polyoxyethylene stearate such as polyoxyl (8) stearate, a polyoxyethylene sorbitan fatty acid ester such as polyoxyethylene (20) sorbitan monostearate, or poloxamers; and a colourant.

The effervescent substance can additionally contain fillers in an amount preferably of 15% by weight to 30% by weight and especially of 26.2% (based on the total weight of the effervescent preparation). Examples of such fillers are lactose and/or sorbitol, either in untreated form or in pretreated form, e.g. granulated form. The effervescent substance can additionally contain a binder in an amount preferably ranging from 0.2% by weight to 0.6% by weight and especially of 0.43% (based on the total weight of the effervescent preparation), a preferred binder being polyvinylpyrrolidone (PVP), preferably in an amount of 0.6% by weight to 1% by weight, based on the effervescent substance, and a colourant.

The carbonate compound, preferably siliconized sodium hydrogen carbonate, is preferably prepared separately by adding silicone antifoam emulsion to sodium hydrogen carbonate (typically about 0.32% by weight, based on the weight of the sodium hydrogen carbonate), the molar ratio of sodium hydrogen carbonate to acid preferably being about 1:1.

An efficient, long-lasting masking of the bitter taste of the active ingredient is achieved by using the aforementioned taste masking agents belonging to the class of substances comprising flavonoids and derivatives thereof and/or polypeptides and derivatives thereof, as described above.

Optimal taste masking is achieved by a combination of two or more sweeteners. It is thus possible, for example, to use a combination of two sweeteners, such as a flavonoid derivative, e.g. neohesperidin, and a polypeptide, e.g. L-aspartyl-D-alanine. An optionally additional, spontaneous, short-lived sweetness can be achieved e.g. with sodium saccharinate, mannitol, maltitol and/or sorbitol, preferably sodium saccharinate. A slow-developing, longer-lasting sweetness can be achieved with aspartame, xylitol and/or alitame, preferably aspartame.

Especially peroral forms of administration that give a taste in the mouth, such as effervescent preparations, suspensions, tablets that disintegrate in the mouth, and in general any other forms of administration, can contain taste correctors such as ethereal oils; organic acids, e.g. citric acid or ascorbic acid; polysaccharides, e.g. sucrose, fructose, glucose, dextrose, isomaltose, inulin, lactitol or trehalose; synthetic sugar substitutes and salts thereof, such as xylitol, cyclamate, acesulfame, sucralose, saccharin, alitame or aspartame; polyols, e.g. glycerol, sorbitol or mannitol; and vanillin.

The production of capsules, such as hard gelatin capsules and soft gelatin capsules, liquid dosage forms, such as solutions, emulsions and suspensions, and rectal products, such as different types of suppositories, e.g. suspension suppositories and dissolving suppositories, or rectal capsules, is known per se and can be carried out with additives known per se.

The production of hard gelatin capsules and soft gelatin capsules is known per se, the composition according to the invention being filled into hard gelatin capsules, preferably as granules, in a manner known per se, or made up into soft gelatin capsules, preferably as a viscous suspension or a paste. Such granules, viscous suspensions and pastes can easily be prepared by those skilled in the art in accordance with analogous formulations.

In one preferred embodiment of pellets which can be used according to the invention, said pellets contain (a) at least one of the active ingredients indomethacin and acemetacin or a mixture thereof in micronized form, preferably obtained by mechanical means, and additionally a binder and a loading agent, and optionally (b) these pellets are coated with a varnish resistant to gastric juice and/or have been produced in the presence of a varnish resistant to gastric juice. These pellets have an apparent density preferably of 1.4-2.4 g/cm³ and particularly preferably of 1.5-1.8 g/cm³. Their diameter preferably ranges from 0.2 to 1.8 mm, the pellet preparation containing preferably about 0.1-80% by weight of active ingredient and preferably about 20-95% by weight and particularly preferably about 40-80% by weight of loading agent, as well as binder, colourant and acidifying agent ad 100% by weight. Examples of particularly suitable loading agents are titanium dioxide, barium sulfate and/or iron oxide.

The forms of administration produced from the pellets according to the invention, such as those mentioned herein, e.g. tablets or capsules, preferably contain the active ingredient(s) in an amount of 25 to 200 mg of active ingredient per unit form of administration, i.e. in amounts of e.g. 25 mg, 30 mg, 50 mg, 60 mg, 80 mg, 90 mg, 100 mg, 180 mg or 200 mg per unit form of administration.

The pellets according to the invention are produced e.g. by mixing at least one of said active ingredients in micronized form with the additives, pelleting the mixture in a manner known per se and optionally providing the resulting pellets with a coating, which may be a varnish coating resistant to gastric juice.

The production of liquid dosage forms, such as solutions, emulsions and suspensions, is known from analogous formulations. Solutions mentioned here according to the relevant literature are molecularly disperse solutions with average particle sizes below 1 nm (1 nanometre) (particles <1 nm). Colloidally disperse solutions are liquid systems with an average particle size of 1 nm-1 μm.

Additives for liquid forms of administration, such as solutions, are known per se, examples being solubilizers such as alcohols, preferably ethanol; surfactants known per se; viscosity builders such as celluloses and cellulose derivatives like HPMC; polyvinyl compounds such as polyvinyl alcohols, polyvinylpyrrolidones and polyvinyl acetate-phthalates; polyethylene glycols and polyethylene oxides; polysaccharides such as polydextrose; mucoadhesives such as carrageenan and chitosan; silicates; alginates such as alginic acid and sodium alginate; and preservatives. Those skilled in the art can easily use and optimize these substances in the necessary concentrations and compositions. Syrups often contain up to 65% by weight of sugar, e.g. sucrose, as well as taste correctors.

Examples of preferred auxiliary substances or additives for emulsions are as follows: surfactants such as anionic surfactants, bile acid salt, preferably sodium glycocholate, and gum arabic; cationic and non-ionic surfactants known per se; higher fatty alcohols such as cetyl alcohol and stearyl alcohol; partial fatty acid esters of polyhydric alcohols, such as ethylene glycol monostearate; partial fatty acid esters of sorbitan (0.01-15%, based on the total weight of the formulation), e.g. sorbitan monolaurate; partial fatty acid esters of polyoxyethylene sorbitan (0.1-15%, based on the total weight of the formulation), e.g. polyoxyethylene sorbitan fatty acid esters; macrogol glycerol fatty acid esters such as macrogol glycerol laurate; fatty acid esters of polyoxyethylene (0.5-10%, based on the total weight of the formulation); fatty alcohol ethers of polyoxyethylene, e.g. polyoxyethylene stearyl ether (0.5-25%, based on the total weight of the formulation); and fatty acid esters of polyglycerol, e.g. polyglycerol oleate. Lecithin is preferred among the amphoteric emulsifiers. In suspensions, surfactants are preferred as dispersants. The concentrations used are known to those skilled in the art from analogous applications.

Preferred examples of rectal products are suspension suppositories and dissolving suppositories. Their production is known per se. Thus the suppository mass is preferably prepared using cacao butter and derivates thereof as triglycerides; hard fats such as mono-, di- and triglycerides of saturated fatty acids, like those of coconut fat or palm kernel fat; and water-soluble compounds such as polyethylene glycols and glyceryl gelatin. Waxes, bentonites or silicon oxides, for example, can be used as viscosity builders. Solubilizers are emulsifiers such as lecithin. The rectal products are produced by the casting (melting) processes and pressing processes known per se. The Examples which follow illustrate the invention.

### EXAMPLE 1 (PREPARATION OF MICRONIZED INDOMETHACIN AND ACEMETACIN)

Coarse indomethacin and acemetacin, both of pharmaceutical grade, were micronized with the Chrispro® Jet-Mill MC 300KX-TD apparatus from Micro-Macinazione SA according to the following grinding principle: The particles of grinding material are caused to collide with one another at high speed in the grinding region by means of lateral nozzles (supply pressure 6.0 bar; working pressure 4.0 bar; nozzle angle 32° 5′). The grinding material is separated into fine and coarse material by centrifugal action. The fine material is discharged through the central outlet and the coarse material is comminuted further until it has all reached the required degree of comminution. The powder obtained had the particle size distribution shown in Table 1:

TABLE 1

| Cumulative distribution | Particle |
|---|---|
| (%) | size (μm) |
| 99 | 20.5 |
| 95 | 15.0 |
| 90 | 12.3 |
| 85 | 10.5 |
| 80 | 9.0 |
| 70 | 7.1 |
| 50 | 3.87 |
| 10 | 0.9 |

### EXAMPLE 2 (PRODUCTION OF A TABLET)

The micronized acemetacin or indomethacin prepared in Example 1, 4 times the amount of modified lactose (spray-dried for direct tableting) and 0.3% by weight of thaumatin (based on the total composition) are passed through a 0.8 mm sieve and then mixed. 0.25% by weight of magnesium stearate is added and mixing is then repeated. The resulting pulverulent mixture is then compressed to tablets on a rotary press.

### EXAMPLE 3 (PRODUCTION OF GRANULES)

The micronized acemetacin or indomethacin prepared in Example 1 is mixed with 15% of citric acid and 74.6% of sodium sulfate (based in each case on the total weight of the granules). The colourant (0.075% by weight, based on the total composition) is dissolved in three times the amount of water, and macrogol glycerol hydroxystearate (1.5% by weight, based on the total composition) is dissolved in 2.5 times the amount of ethanol. The above-mentioned pulverulent mixture is moistened with the ethanolic solution, and the aqueous colourant solution is then added. Sufficient 1:1 ethanol/water mixture is added to achieve uniform moistening and the mixture is then granulated through a sieve (2.0 mm). The moist granules are dried for approx. 2 hours at 50° C. down to a loss on drying of less than 0.3% by weight, and then passed through a sieve of mesh size 1.0 mm.

### EXAMPLE 4 (PULVERULENT MIXTURE FOR HARD GELATIN CAPSULES)

The micronized acemetacin or indomethacin prepared in Example 1 is mixed with lactose (200 mesh, 17% by weight, based on the total composition) and granulated lactose (35.7% by weight, based on the total composition). After the addition of 2% by weight of talc and 2% by weight of magnesium stearate (based on the total composition) and 0.35% by weight of silicon dioxide (based on the total composition), the pulverulent mixture is filled into hard gelatin capsules.

### EXAMPLE 5 (PRODUCTION OF TABLETS)

Polyvinylpyrrolidone (1% by weight, based on the total composition) is dissolved in 70 times the amount of ethanol. The micronized acemetacin or indomethacin prepared in Example 1 is mixed with lactose (46% by weight, based on the total composition), 0.2% by weight of neohesperidin and 0.2% by weight of thaumatin, and the mixture is granulated with the ethanolic polyvinylpyrrolidone solution and then dried at 40° C. down to a residual moisture content of 5% by weight. 12.4% by weight (based on the total composition) of talc, 2.7% by weight of wheat starch and 1.5% by weight of stearic acid are added to these granules and the whole is mixed. After the addition of 1% by weight of magnesium stearate (based on the total composition) and further mixing, the total composition is compressed to tablets on a rotary press.

### EXAMPLE 6 (PRODUCTION OF AN EFFERVESCENT TABLET)

(a) The effervescent substance: 32.5% by weight of anhydrous citric acid (% by weight based in each case on the total composition), 11.4% by weight of sodium hydrogen carbonate, 19.8% by weight of lactose monohydrate, 6.5% by weight of sorbitol and neohesperidin DC (0.3% by weight), 0.6% by weight of aspartame and 0.12% by weight of saccharin sodium are charged into the granulator and intimately mixed. Yellow-orange (0.02% by weight) is dissolved in 3 times the amount of water at 50° C. Polyvinylpyrrolidone (0.4% by weight) is dissolved in approx. 5 times the amount of 96% ethanol. The above-mentioned pulverulent mixture is moistened with this ethanolic solution. The colourant solution is added immediately afterwards. Further ethanol (96%) is added until uniform moistening is achieved, and the mixture is then granulated through a 2.0 mm sieve. The moist granules are dried at 60° C. down to a loss on drying of 0.3% by weight, and then passed through a 1.25 mm sieve.

(b) Sodium hydrogen carbonate, siliconized: Sodium hydrogen carbonate (1.3% by weight, based on the total composition) is placed in a wet mixer. With constant stirring, the appropriate amount (0.005%, based on the total composition) of silicone antifoam emulsion (33%) is slowly added to this dry substance. Stirring is continued for about 30 minutes and the mixture is then dried on trays at 55° C. and passed through a 0.7 mm sieve.

(c) The effervescent tablet: The above-described effervescent substance [section (a)], the above-described siliconized sodium hydrogen carbonate [section (b)], the pellets produced in Example 3 (20.6% by weight, based in each case on the total composition) and 1.8% by weight of sodium hydrogen carbonate, 3.1% by weight of polyethylene glycol 6000 and 1.5% by weight of lemon flavouring are mixed for 15 minutes in a suitable tumbling mixer. The finished mixture is compressed to effervescent tablets on a rotary press at a maximum relative humidity of 25% and at 20° C.

### EXAMPLE 7 (PRODUCTION OF AN EFFERVESCENT POWDER)

(a) The effervescent substance: 32.5% by weight of anhydrous citric acid (% by weight based in each case on the total composition), 11.4% by weight of sodium hydrogen carbonate, 19.5% by weight of lactose monohydrate, 6.5% by weight of sorbitol and neohesperidin DC (0.3% by weight), thaumatin (0.3% by weight), 0.6% by weight of aspartame and 0.12% by weight of saccharin sodium are charged into the granulator and intimately mixed. Yellow-orange (0.02% by weight) is dissolved in 3 times the amount of water at 50° C. Polyvinylpyrrolidone (0.4% by weight) is dissolved in approx. 5 times the amount of 96% ethanol. The above-mentioned pulverulent mixture is moistened with this ethanolic solution. The colourant solution is added immediately afterwards. Further ethanol (96%) is added until uniform moistening is achieved, and the mixture is then granulated through a 2.0 mm sieve. The moist granules are dried at 60° C. down to a loss on drying of 0.3% by weight, and then passed through a 1.25 mm sieve.

(b) Sodium hydrogen carbonate, siliconized: Sodium hydrogen carbonate (1.3% by weight, based on the total composition) is placed in a wet mixer. With constant stirring, the appropriate amount (0.005%, based on the total composition) of silicone antifoam emulsion (33%) is slowly added to this dry substance. Stirring is continued for about 30 minutes and the mixture is then dried on trays at 55° C. and passed through a 0.7 mm sieve.

(c) Finished effervescent powder: The above-described effervescent substance [section (a)], the above-described siliconized sodium hydrogen carbonate [section (b)], the pellets produced in Example 3 (20.6% by weight, based in each case on the total composition) and 1.8% by weight of sodium hydrogen carbonate, 3.1% by weight of polyethylene glycol 6000 and 1.5% by weight of lemon flavouring are mixed for 15 minutes in a suitable tumbling mixer. The finished mixture is packaged as a single dose of effervescent powder, e.g. in a sachet, at a maximum relative humidity of 25% and at 20° C.

### EXAMPLE 8 (PRODUCTION OF EFFERVESCENT TABLETS AND EFFERVESCENT POWDER)

Analogously to Example 6 for the production of an effervescent tablet, and analogously to Example 7 for the production of effervescent powder, effervescent tablets and effervescent powder can be produced with the compositions indicated in Table 2 using micronized acemetacin. The indicated amount of acemetacin can be changed to the corresponding amount of indomethacin.

TABLE 2

| | Formulation 1 | Formulation 2 |
|---|---|---|
| | (mg) | (mg) |
| Acemetacin (active ingredient) | 60.00 | 60.00 |
| Auxiliary substances: | | |
| Citric acid, anhydrous | 1156.00 | 1156.00 |
| Sodium hydrogen carbonate | 470.34 | 470.34 |
| Sodium sulfate | 500.00 | 500.00 |
| Alpha-lactose · 1H₂O | 642.30 | 642.30 |
| Sorbitol | 212.00 | 212.00 |
| Yellow-orange 85 E 110 | 1.20 | 1.20 |
| Macrogol glycerol hydroxystearate | 10.00 | 10.00 |
| Polyvidone | 14.00 | 14.00 |
| Neohesperidin DC, E 959 | 10.00 | —.— |
| Thaumatin, E 957 | —.— | 10.00 |
| Aspartame | 20.00 | 20.00 |
| Saccharin sodium | 4.00 | 4.00 |
| Lemon flavouring, Evogran, | 50.00 | 50.00 |
| 301686, Symrise | | |
| Polyethylene glycol 6000 | 100.00 | 100.00 |
| Silicone antifoam emulsion | 0.16 | 0.16 |

### EXAMPLE 9 (PRODUCTION OF SUPPOSITORIES)

The hard fats Witepsol W 35 (56.2 parts) and Witepsol E 75 (38.5% by weight) are melted at 55° C., with stirring. Cetostearyl alcohol (2.9 parts) is then melted at 75° C. and admixed lege artis to the above melt. Acemetacin or indomethacin micronized according to Example 1 (58 parts in each case) is separately stirred into the melt obtained and rinsed with the remaining melt, after which the resulting mixture is cooled to 40° C. Suppositories are then filled into printed strips using the suppository casting machine, with constant stirring. The suppositories are subsequently cooled firstly to room temperature then slowly to 15° C. and the strips are heat-sealed.

### EXAMPLE 10 (PRODUCTION OF AN INTRAMUSCULAR INJECTION)

After prior sterilization, micronized acemetacin or indomethacin prepared according to Example 1 is separately incorporated into 30 times the amount of medium-chain triglycerides by stirring. The resulting formulation is filled into vials and sealed. All operations are carried out under aseptic conditions.

Translator's Report
Your order ref.: P1740PCT Date: 24 Jan. 2008

| Page | Para | Line | Comment |
|---|---|---|---|
| 3 | | 9 | Doduenum = Duodenum |
| 4 | | 7 | Verfahren = ein Verfahren |
| 6 | | 19 | welchem = welchen |
| 7 | | 22 | Hesperitin = Hesperetin |
| 7 | | 26 | entsprechend = entsprechen |
| 8 | | 2 | einem = einen |
| | | | der = den |
| 11 | | 9 | wird = werden |
| 11 | | 26 | Brausekörper = Brausekörpers |
| 12 | | 25 | einen = einem |
| 13 | | 8 | Saccharose and sucrose are synonyms. |
| 13 | | 9 | Isomalt = Isomaltose? |
| 14 | | 2 | einem = einen |
| 15 | | 6 | Saccharose and sucrose are synonyms. |
| 15 | | 7 | Isomalt = Isomaltose? |
| 16 | | 5 | magensaftresisten = magensaftresistenten |
| 16 | | 8 | Delete ‘vorzugsweise 1.5-1.8 g/cm³,’ (repetition). |
| 17 | | 14 | Polyvinylverbinungen = Polyvinylverbindungen |
| 17 | | 31 | Ethylenmonostearat = Ethylenglycolmonostearat? |
| 23 | | 1 | in = wird in |
| 24 | Table 2 | | column 1, row 15: Sacharin = Saccharin |
| 25 | | 1 | erhaltenen = erhaltene |
| 25 | | 2 | It is not clear where the remaining melt comes from. |
| 25 | | 5 | dann = werden dann |
| 28 | | 14 | Hesperitin = Hesperetin |
| 28 | | 16 | Delete ‘darstellt’? |
| 29 | | 7 | Delete ‘sowie’? |
| 30 | | 1 | nen = nem |
| 30 | | 17 | Delete repetition of ‘nach’. |
| 31 | | 2.3 | Delete ‘vorzugsweise 1.5-1.8 g/cm³,’ (repetition). |

Note:
Minor errors are rectified without comment. Material errors are rectified except in the case of literal translations, when they are reproduced where possible.

1. A pharmaceutically effective composition consisting of:
a mixture with at least one flavonoid derivative selected from the group consisting of: chalcones glycosides and dihydrochalcones glycosides, and combinations thereof; and
at least one of the active ingredients indomethacin and acemetacin, characterized in that
(i) the active ingredient or a mixture of these active ingredients is/are in micronized form, whereby said micronized form has been obtained by micronization using mechanical means;
(ii) the micronized active ingredient has a particle size distribution in the range of 0.1 μm (micron) to 100 μm (micron); and
(iii) said active ingredient in micronized form being present in the form of microcrystals;
wherein said composition is in a pharmaceutical form for oral administration.

2. Composition according to claim 1, characterized in that the micronized active ingredient or mixture of micronized active ingredients has been obtained by micronization using dry grinding, jet milling or wet grinding.

3. Composition according to claim 1, characterized in that it is in the form of (i) tablets, (ii) capsules, or (iii) a liquid dosage form.

4. Composition according to claim 1, characterized in that the pharmaceutical form of oral administration is in the form of bulk powders, pellets, peroral tablets, chewing tablets, oral tablets, dissolving tablets or effervescent tablets; filled in hard gelatin capsules or soft gelatin capsules; or a solution, emulsion or suspension.

5. Composition according to claim 1, characterized in that at least 90% by volume of the active ingredient, has a mean particle size distribution below 25 μm (micron) and the lower limit of the particle size distribution is about 0.1 μm (micron).

6. Composition according to claim 1, characterized in that at least 50% by volume of the active ingredient has a mean particle size distribution below 10 μm (micron), and the lower limit of the particle size distribution is about 0.1 μm (micron).

7. Composition according to claim 1, characterized in that at least 30% by volume of the active ingredient has a mean particle size distribution below 5 μm (micron) and the lower limit of the particle size distribution is about 1 μm (micron).

8. Composition according to claim 1, characterized in that the flavonoid derivatives are selected from the group comprising naringin chalcone, hesperetin dihydrochalcone glucoside and, neohesperidin dihydrochalcone.

9. Composition according to claim 1, characterized in that the weight ratio of active ingredient to flavonoid compound ranges from 10:1 to 50:1.

10. Composition according to claim 1 in the form of an effervescent tablet, characterized in that it consists of (a) active ingredient granules containing the micronized active ingredient, the latter being (b) in a mixture with at least one flavonoid derivative, (c) an effervescent substance consisting of at least one siliconized inorganic carbonate compound or bicarbonate compound and an organic acid, and optionally (d) other additives.

11. Composition according to claim 10, characterized in that the additives which may be present are selected from the group comprising sweeteners, synthetic sugar substitutes and salts thereof, polyols, natural and synthetically prepared flavourings, loading agents, surfactants, colourants, fillers and binders.

12. Composition according to claim 1, characterized in that it is in the form of pellets and (a) these pellets contain at least one of the active ingredients indomethacin and acemetacin or a mixture thereof in micronized form, and additionally a binder and a loading agent, and optionally (b) these pellets have been coated with a varnish resistant to gastric juice and/or granulated in the presence of a varnish resistant to gastric juice.

13. Composition according to claim 12, characterized in that the pellets have an apparent density of 1.4-2.4 g/cm³, and their diameter ranges from 0.2 to 1.8 mm.

14. Composition according to claim 4, characterized in that the pellets contain about 0.1-80% by weight of active ingredient, by weight of loading agent, as well as binder, colourant and acidifying agent ad 100% by weight.

15. A form of administration produced from the composition according to claim 1 consisting of tablets or capsules, containing the active ingredient(s) in an amount of 25 mg to 200 mg of active ingredient per unit form of administration.

16. An active ingredient consisting of indomethacin and acemetacin as bulk powders, optionally in a mixture with other additives, suitable for the preparation of a composition according to claim 1, characterized in that they are in a micronized form obtained by mechanical means.

17. A method for a medicinal treatment comprising the steps of:
providing a composition according to claim 16 to an individual for said medical treatment of chronic and acute pain conditions, inflammations and fever, especially chronic polyarthritis, degenerative joint diseases, especially of the large joints and the spinal column, Bechterew's disease, gout, inflammatory conditions of the joints, muscles and tendons, tendovaginitis, bursitis, lumbago and superficial venous inflammations (thrombophlebitis).

18. Composition according to claim 4, characterized in that the pellets contain about 20-95% by weight of active ingredient, by weight of loading agent, as well as binder, colourant and acidifying agent ad 100% by weight.

19. A pharmaceutically effective composition consisting of:
a mixture with at least one flavonoid derivative selected from the group consisting of: chalcones glycosides and dihydrochalcones glycosides, and combinations thereof;
a polypeptide or a mixture of polypeptides; and
at least one of the active ingredients indomethacin and acemetacin, characterized in that
(i) the active ingredient or a mixture of these active ingredients is/are in micronized form, whereby said micronized form has been obtained by micronization using mechanical means;
(ii) the micronized active ingredient has a particle size distribution in the range of 0.1 μm (micron) to 100 μm (micron); and
(iii) said active ingredient in micronized form being present in the form of microcrystals;
wherein said composition is in a pharmaceutical form for oral administration.
